(19) United States
(12) Patent Application Publication
(10) Pub. No.: US 2013/0090321 A1
(43) Pub. Date: Apr. 11, 2013

(54) METHOD FOR TREATING BRAIN CANCER

(71) Applicants: Hooshmand SHESHBARADARAN, Hoboken, NJ (US); Rebecca BAERGA, Hoboken, NJ (US); Jenel COBB, Hoboken, NJ (US); Mojtaba Seied VALIAHDI, Vienna (AT); Bernhard KEPPLER, Vienna (AT)

(72) Inventors: Hooshmand SHESHBARADARAN, Hoboken, NJ (US); Rebecca BAERGA, Hoboken, NJ (US); Jenel COBB, Hoboken, NJ (US); Mojtaba Seied VALIAHDI, Vienna (AT); Bernhard KEPPLER, Vienna (AT)

(73) Assignee: NIIKI PHARMA INC., Hoboken, NJ (US)

(21) Appl. No.: 13/594,941

(22) Filed: Aug. 27, 2012

Related U.S. Application Data
(63) Continuation of application No. PCT/US2011/026144, filed on Feb. 25, 2011.
(60) Provisional application No. 61/414,882, filed on Nov. 17, 2010.
(60) Provisional application No. 61/308,813, filed on Feb. 26, 2010.

(51) Int. Cl. A61K 31/555; C07F 5/00; A61K 31/4188

(52) U.S. Cl. CPC A61K 31/555; A61K 31/4188; C07F 5/003
USPC 514/187

(57) ABSTRACT

Methods and compositions for treating brain cancer are disclose including refractory brain cancer.

### CROSS-REFERENCE TO RELATED APPLICATIONS

This application is a continuation of PCT/US11/26144, and claims the benefit of U.S. Provisional Application No. 61/308,813 filed on Feb. 26, 2010 and U.S. Provisional Application No. 61/414,882 filed on Nov. 17, 2010, the entire content of all of which are incorporated herein by reference.

### FIELD OF THE INVENTION

The present invention generally relates to pharmaceutical compositions and methods for treating cancer, and particularly to a pharmaceutical composition having tris(8-quinolinolato) gallium(III), and method of using thereof.

### BACKGROUND OF THE INVENTION

It is estimated that there are over 40,000 new cases of brain cancer every year in the United States alone, and more than 13,000 die each year from the disease. Aside from surgery and radiation therapy, there are very few treatment options. Temozolomide and nitrosourea are the only accepted chemotherapeutics for brain cancer, and yet have shown rather limited effectiveness. Thus, there is a significant need for new agents in treating brain cancer.

US Patent Publication No. 2009/0137620 discloses that the compound tris(8-quinolinolato)gallium(III) has been shown to be effective in causing apoptosis and cell death in melanoma cell lines. However, it is unknown whether the compound is useful in treating brain cancer, especially those refractory to other anti-cancer drugs.

### SUMMARY OF THE INVENTION

The present invention provides methods of treating brain cancer. In one aspect, the present invention provides a method of treating, preventing or delaying the onset of, brain cancer comprising administering to a patient having brain cancer a therapeutically or prophylactically effective amount of a compound according to Formula (I) below or a pharmaceutically acceptable salt thereof (e.g., tris(8-quinolinolato)gallium(III)).

In accordance with another aspect, a method of treating, preventing or delaying the onset of a refractory brain cancer is provided comprising administering a therapeutically or prophylactically effective amount of a compound according to Formula (I) below or a pharmaceutically acceptable salt thereof (e.g., tris(8-quinolinolato)gallium(III)) to a patient refractory to a treatment comprising at least one of nitrosourea (e.g., BCNU) and temozolomide.

In yet another aspect, a method of treating, preventing or delaying the onset of brain cancer is provided comprising administering to a cancer patient in need of treatment, simultaneously or sequentially, a therapeutically effective amount of (1) a compound according to Formula (I) below or a pharmaceutically acceptable salt thereof (e.g., tris(8-quinolinolato)gallium(III)), and (2) temozolomide. In specific embodiments, the combination is used for the treatment of glioblastoma or astrocytoma.

Use of the compound according to Formula (I) below or a pharmaceutically acceptable salt thereof (e.g., tris(8-quinolinolato)gallium(III)) for the manufacture of a medicament for use in the methods of the present invention is also provided.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF DRAWINGS

FIG. 1 is a graph showing the dose-dependent growth inhibition by tris(8-quinolinolato)gallium(III) (MTT assay) in a 3-dimensional tumor model (HuBiogel, Vivo Biosciences, Birmingham, Ala.) derived from glioma cell line U87. X axis is drug concentration in μM and Y axis is percentage of control;

FIG. 2 is a combination index plot illustrating the additive to synergistic activity between tris(8-quinolinolato)gallium(III) and temozolomide in the glioblastoma cell line U251.

### DETAILED DESCRIPTION OF THE INVENTION

The present invention is at least in part based on the discovery that the compound tris(8-quinolinolato)gallium(III) is especially effective in treating brain cancers. Accordingly, in accordance with a first aspect of the present invention, a method is provided for treating brain cancer. The method comprises treating a brain cancer patient in need of treatment with a therapeutically effective amount of a gallium complex of Formula (I)

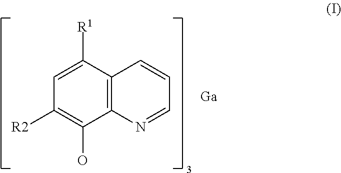

wherein R¹ represents hydrogen, a halogen or a sulfono group SO₃M, in which M is a metal ion, and R2 represents hydrogen, or R¹ is Cl and R² is I, or a pharmaceutically acceptable salt thereof. Brain tumor is an intracranial solid neoplasm within the brain or the central spinal canal. In one embodiment, the method for treating brain cancer comprises treating a brain cancer patient in need of treatment with a therapeutically effective amount of compound of Formula (I) or a pharmaceutically acceptable salt thereof, wherein the brain cancer is not glioblastoma. That is, the present invention is directed to the use of an effective amount of a compound according to Formula (I) or a pharmaceutically acceptable salt thereof for the manufacture of medicaments for treating a brain cancer in patients identified or diagnosed as having a brain cancer, optionally said brain cancer not being glioblastoma.

In the various embodiments of this aspect of the present invention, the treatment method optionally also comprises a step of diagnosing or identifying a patient as having brain tumor. The identified patient is then treated with or administered with a therapeutically effective amount of a compound of the present invention, e.g., tris(8-quinolinolato)gallium(III) which has a formula:

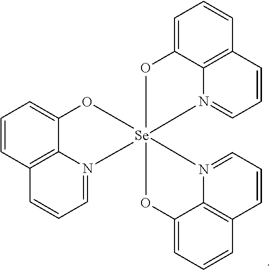

Various brain cancers can be diagnosed in any conventional diagnostic methods known in the art including MRI scan, CAT scan, PET scan, biopsy, etc.

In addition, it has also been surprisingly discovered that the compound tris(8-quinolinolato)gallium(III) is equally effective in brain cancer cells resistant to nitrosourea (e.g., bis-chloronitrosourea (BCNU)) or temozolomide. Accordingly, another aspect of the present invention provides a method of treating refractory brain cancer comprising treating a patient identified as having refractory brain cancer with a therapeutically effective amount of a compound of Formula (I) or a pharmaceutically acceptable salt thereof (e.g., tris(8-quinolinolato)gallium(III)). In one embodiment, the patient has a brain cancer that is refractory to a treatment comprising a nitrosourea drug such as BCNU. In another embodiment, the patient has a brain cancer that is refractory to a treatment comprising temozolomide. That is, the present invention is also directed to the use of a compound of Formula (I) or a pharmaceutically acceptable salt thereof (e.g., tris(8-quinolinolato)gallium(III)) for the manufacture of medicaments for treating refractory brain cancer, e.g., a brain cancer refractory to nitrosourea and/or temozolomide.

The term “refractory brain cancer,” as used herein refers to a brain cancer that either fails to respond favorably to an anti-neoplastic treatment that does not include a compound of Formula (I), or alternatively, recurs or relapses after responding favorably to an antineoplastic treatment that does not include a compound of Formula (I). Accordingly, “a brain cancer refractory to a treatment” as used herein means a brain cancer that fails to respond favorably to, or resistant to, the treatment, or alternatively, recurs or relapses after responding favorably to the treatment.

Thus, in some embodiments, in the method of the present invention, a compound of Formula (I) or a pharmaceutically acceptable salt thereof (e.g., tris(8-quinolinolato)gallium(III)) is used to treat brain cancer patients having a tumor previously treated with a treatment regimen comprising one or more drugs such as nitrosourea (e.g., BCNU) and temozolomide. For example, the method is used to treat a brain cancer patient having previously been treated with a treatment regimen that includes one or more drugs such as nitrosourea (e.g., BCNU) and temozolomide, and whose brain cancer was found to be non-responsive to the treatment regimen or have developed resistance to the treatment regimen. In other embodiments, the method is used to treat a brain cancer patient previously treated with a treatment comprising one or more drugs such as nitrosourea (e.g., BCNU) and temozolomide, but the brain cancer has recurred or relapsed, that is, a brain cancer patient who has previously been treated with one or more such drugs, and whose cancer was initially responsive to the previously administered one or more such drugs, but was subsequently found to have relapsed.

In specific embodiments, a compound of Formula (I) or a pharmaceutically acceptable salt thereof (e.g., tris(8-quinolinolato)gallium(III)) is used to treat brain cancer patients previously treated with a nitrosourea (e.g., BCNU).

In yet other specific embodiments, a compound of Formula (I) or a pharmaceutically acceptable salt thereof (e.g., tris(8-quinolinolato)gallium(III)) is used to treat brain cancer patients previously treated with temozolomide, i.e., who have a brain cancer that exhibits resistance to, or relapsed after, a treatment including, temozolomide.

To detect a refractory brain cancer, patients undergoing initial treatment can be carefully monitored for signs of resistance, non-responsiveness or recurring brain cancer. This can be accomplished by monitoring the patient's cancer's response to the initial treatment which, e.g., may include nitrosourea or temozolomide. The response, lack of response, or relapse of the cancer to the initial treatment can be determined by any suitable method practiced in the art. For example, this can be accomplished by the assessment of tumor size and number. An increase in tumor size or, alternatively, tumor number, indicates that the tumor is not responding to the chemotherapy, or that a relapse has occurred. The determination can be done according to the “RECIST” criteria as described in detail in Therasse et al. J. Natl. Cancer Inst. 92:205-216 (2000).

In accordance with yet another aspect of the present invention, a method is provided for preventing or delaying the onset of brain cancer, or preventing or delaying the recurrence of brain cancer, which comprises treating a patient in need of the prevention or delay with a prophylactically effective amount of a compound of Formula (I) or a pharmaceutically acceptable salt thereof (e.g., tris(8-quinolinolato)gallium(III)).

For purposes of preventing or delaying the recurrence of brain cancer, brain cancer patients who have been treated and are in remission or in a stable or progression free state may be treated with a prophylactically effective amount of a compound of Formula (I) or a pharmaceutically acceptable salt thereof (e.g., tris(8-quinolinolato)gallium(III))to effectively prevent or delay the recurrence or relapse of brain cancer.

In addition, it has been surprisingly discovered that combination of tris(8-quinolinolato)gallium(III) and temozolomide provided a significant synergy in causing apoptosis in glioblastoma cells. Thus, the present invention also provides a combination therapy method, in which temozolomide and the compound of Formula (I) (e.g., tris(8-quinolinolato)gallium(III)) are used together in the same treatment regimen. That is, the present invention provides a method of treating, preventing or delaying the onset of brain cancer comprising administering to a brain cancer patient in need of treatment, simultaneously or sequentially, a therapeutically effective amount of (1) a compound according to Formula (I) or a pharmaceutically acceptable salt thereof (e.g., tris(8-quinolinolato)gallium(III)), and (2) temozolomide. An effective amount of additional drugs such as bevacizumab, nitrosourea and procarbazine may also be optionally included in the combination therapy and administered to the brain cancer patient. In specific embodiments, the combination is used for the treatment of glioblastoma multiforme (GBM) or anaplastic astrocytoma. In preferred embodiments, the gallium complex is tris(8-quinolinolato)gallium(III) or a pharmaceutically acceptable salt thereof.

The various aspects of the present invention can be useful in various brain malignancies including, but not limited to, acoustic neuroma, astrocytoma (e.g., pilocytic astrocytoma, low-grade astrocytoma, anaplastic astrocytoma), glioblastoma multiforme (GBM) and other gliomas (brain stem glioma, optic nerve glioma, ependymoma, mixed glioma, optic nerve glioma, oligodendroglioma, and subependymoma), chordoma, CNS lymphoma, craniopharyngioma, medulloblastoma, meningioma, pituitary tumors, primitive neuroectodermal (PNET), schwannoma, pineal tumor and rhabdoid tumor.

As used herein, the phrase “treating . . . with . . . ” or a paraphrase thereof means administering a compound to the patient or causing the formation of a compound inside the body of the patient.

In accordance with the method of the present invention, brain cancer can be treated with a therapeutically effective amount of a compound of Formula (I) or a pharmaceutically acceptable salt thereof (e.g., tris(8-quinolinolato)gallium(III)) alone as a single agent, or alternatively in combination with one or more other anti-cancer agents.

The pharmaceutical compounds of Formula (I) can be administered through intravenous injection or oral administration or any other suitable means at an amount of from 0.1 mg to 1000 mg per kg of body weight of the patient based on total body weight. The active ingredients may be administered at predetermined intervals of time, e.g., three times a day. It should be understood that the dosage ranges set forth above are exemplary only and are not intended to limit the scope of this invention. The therapeutically effective amount of the active compound can vary with factors including, but not limited to, the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In accordance with the present invention, it is provided a use of a compound having a compound of Formula (I) or a pharmaceutically acceptable salt thereof (e.g., tris(8-quinolinolato)gallium(III)) for the manufacture of a medicament useful for treating brain cancer alone or in combination with temozolomide. The medicament can be, e.g., in an oral or injectable form, e.g., suitable for intravenous, intradermal, or intramuscular administration. Injectable forms are generally known in the art, e.g., in buffered solution or suspension.

In accordance with another aspect of the present invention, a pharmaceutical kit is provided comprising in a container a unit dosage form of a compound of Formula (I) or a pharmaceutically acceptable salt thereof (e.g., tris(8-quinolinolato)gallium(III)), and optionally instructions for using the kit in the methods in accordance with the present invention, e.g., treating, preventing or delaying the onset of brain cancer, or preventing or delaying the recurrence of brain cancer, or treating refractory brain cancer. In one embodiment, a pharmaceutical kit is provided comprising in a compartmentalized container (1) a unit dosage form of a compound of Formula (I) or a pharmaceutically acceptable salt thereof (e.g., tris(8-quinolinolato)gallium(III)); and (2) a unit dosage form of temozolomide. As will be apparent to a skilled artisan, the amount of a therapeutic compound in the unit dosage form is determined by the dosage to be used on a patient in the methods of the present invention. In the kit, a compound having a compound of Formula (I) or a pharmaceutically acceptable salt thereof (e.g., tris(8-quinolinolato)gallium(III)) can be in a tablet form in an amount of, e.g., 1 mg. Temozolomide to be used in the combination therapy and included in the kit can be in any dosage form generally known or used in the art, e.g., tablet, capsule, a lyophilized form for reconstitution of an injectable form, etc. Optionally, the kit further comprises instructions for using the kit in the combination therapy method in accordance with the present invention.

In accordance with another aspect of the present invention, a pharmaceutical composition is provided, comprising an effective amount of (1) a compound of Formula (I) or a pharmaceutically acceptable salt thereof (e.g., tris(8-quinolinolato)gallium(III)); and (2) temozolomide. The pharmaceutical composition can be in any pharmaceutically acceptable dosage forms including, but not limited to, tablet, capsule, a lyophilized form for reconstitution of an injectable form, solution or suspension, etc. The amount of the drugs to be included in the composition can vary and depend on the amount to be administered to a patient. For example, the compound of Formula (I) or a pharmaceutically acceptable salt thereof (e.g., tris(8-quinolinolato)gallium(III)) can be included in the composition at an amount of from 1 mg to about 1000 mg, and temozolomide can be at 5 mg, 20 mg, 100 mg, 140 mg, 180 mg or 250 mg.

### Example 1

To test the activities of tris(8-quinolinolato)gallium(III) (“drug”), MTT assays were performed using glioblastoma cell lines. Cells were plated (2×10³ cells in 100 μl/well) in 96-well plates and allowed to recover for 24 hours. The drug was added in another 100 μl growth medium and incubated with cultured cells for 3 hours before the cell culture medium was replaced to remove the drug. Cell death was measured 72 hours after the initial incubation by MTT assay following the manufacturer's recommendations (EZ4U, Biomedica, Vienna, Austria). The cell lines tested and IC₅₀ values (drug concentration with 50% growth inhibition) are summarized below in Table 1. Tris(8-quinolinolato)gallium(III) was effective in inducing cell death in all cell lines in Table 1 with IC₅₀ values ranging from about 0.3 μM to about 2.5 μM. Historical data on the inhibition of the cell lines by the nitrosourea drug BCNU are provided in Table 1 below. It is noted that tris(8-quinolinolato)gallium(III) is active against U87 cell line which is relatively resistant to BCNU. In addition, tris(8-quinolinolato)gallium(III) is active in T98G cells which have been shown to be resistant to temozolomide. See Kanzawa et al., J. Neurosurg., 99:1047-1052 (2003).

TABLE 1

| | | BCNU (% |
|---|---|---|
| | | cytostasis |
| | Drug IC₅₀ | with 50 μg/ml |
| Cell Line | (μM) | BCNU)* |
| ACHU | 0.3 | |
| T98G | 2.0 | 37.5 ± 8.6 |
| U87 | 0.94 | 18.5 ± 8.2 |
| U373 | 2.5 | 30.4 ± 8.6 |

*historical data from Bowles et al., J. Neurosurg., 73: 248-253 (1990).

### Example 2

The compound tris(8-quinolinolato)gallium(III) was tested in a 3-dimensional tumor model derived from glioma cell line U87. Specifically, cells were trypsinized, washed, counted by trypan blue exclusion. Tumor beads were then prepared by mixing 20,000 cells/10 μl of HuBiogel (4 mg/mL) (See U.S. patent application Ser. No. 10/546,506, which is incorporated herein by reference). The 3-D tumor beads were cultivated for 72 hours in multi-well plates with complete media (10% FBS) in a 37° C. incubator +5% CO₂. Mini-tumors were treated with various concentrations of the test compound tris(8-quinolinolato)gallium(III) in media (final 0.2-0.3% DMSO) or control (DMSO). Repeated drug treatment was done by removing the culture media and replacing with fresh media with drug compound or DMSO. On Day 3, MTT assay and live-cell staining with Calcein AM were performed (5 beads/assay set).

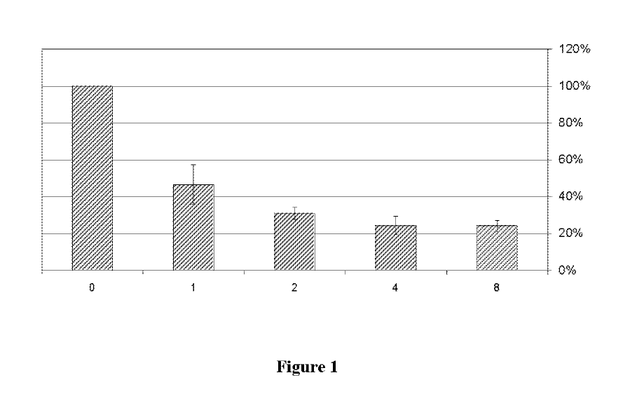

Tris(8-quinolinolato)gallium(III) exhibited dose-dependent tumor killing effective in live-cell staining/image analysis, and significantly inhibited tumor proliferation activity. See FIG. 1. Statistical analysis of data sets (Average, T-test, GI-50) was performed using MS-Excel program. The T-test result is shown in Table 2 below. The average GI-50 (the drug concentration required for growth inhibition at 50%) is 0.94 μM.

TABLE 2

| | Concentration (μM) | | | |
|---|---|---|---|---|
| t-test | 8 | 4 | 2 | 1 |
| HuBiogel (control | 1.12672E−11 | 7.18966E−10 | 3.82139E−11 | 3.60697E−06 |
| vs experiment) | | | | |
| | control vs 8 μM | control vs 4 μM | control vs 2 μM | control vs 1 μM |

### Example 3

### Activities of Tris(8-quinolinolato)gallium(III) in Human Glioblastoma Cell Line Resistant to Temozolomide and BCNU

Tris(8-quinolinolato)gallium(III) and temozolomide were tested in human glioblastoma cell line U251 (which is resistant to BCNU, See Beljanski et al., Anticancer Res., 13(6A):2301-8 (1993)). Specifically, ATCC's MTT Cell Proliferation Assay® was performed using glioblastoma cell line U251. Stock cultures were allowed to grow to 70-80% confluence for this study. The anti-proliferative activity of tris(8-quinolinolato)gallium(III) or temozolomide against the cell line was evaluated in vitro using the ATCC's MTT Cell Proliferation Assay (Catalog No. 30-1010K). Cultures were maintained in a 37° C. humidified 5% CO₂/95% air atmosphere. The cells were treated with tris(8-quinolinolato)gallium(III) or temozolomide at 1,000 μM, or a series of 4× dilutions thereof (250 μM, 62.5 μM, etc.). 100 μl of medium was removed from each well at 72 hours post-treatment and 10 μl MTT reagent was added to each well. The plates were incubated at 37° C. for 4 hours and then 100 μl of detergent was added. The plates were left overnight at room temperature in the dark and was read on a plate reader using SoftMax® Pro (version 5.2, Molecular Devices).

The absorbance data was analyzed as follows: Absorbance values were converted to Percent of Control and plotted against test agent concentrations for IC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration versus Percent of Control were analyzed using the 4-parameter equation to obtain IC₅₀ values and other parameters that describe the sigmoidal dose response curve.

The IC₅₀ value for the test agent was estimated by curve-fitting the data using the following four parameter-logistic equation:

$$Y = \frac{Top - Bottom}{1 + {( X / {IC}_{50} )}^{n}} + Bottom$$

wherein “Top” is the maximal % of control absorbance (100%), “Bottom” is the minimal % of control absorbance at the highest agent concentration (down to zero), Y is the Percent of Control absorbance, X is the test agent Concentration, IC₅₀ is the concentration of agent that inhibits cell growth by 50% compared to the control cells, n is the slope of the curve.

The IC₅₀ of tris(8-quinolinolato)gallium(III) in the U251 cell line was 4.04 μM whereas the IC₅₀ of temozolomide was 283 μM and that of BCNU was 53.7 μM. Thus, tris(8-quinolinolato)gallium(III) is effective in glioblastoma cells resistant to temozolomide or BCNU.

### Example 4

### Combination Studies

Human glioblastoma cell line U251 cells were cultured under conditions described in Example 3. The cells were sub-cultured regularly to maintain log phase growth. On the day of EC₅₀ plate seeding, the cells were processed and seeded into 96-well cell culture-treated plates one cell line at a time. The cells were removed from their culture flasks using trypsin solution pooled in a sterile conical tube and centrifuged at 350 xg for 5 minutes at room temperature. Pelleted cells were re-suspended in complete media and then counted with a Neubauer Bright-Line® hemacytometer and trypan blue viability stain. The cell suspensions were diluted (based on live cell counts) using complete media to yield a final suspension density (cells/ml) based on previously determined seeding densities for each cell line for a 72 hour 96-well plate assay. The tissue culture treated plates for EC₅₀ testing were seeded at 1.5×10³ cells/well, and incubated overnight at 37° C. in a 5% CO₂, 95% air humidified atmosphere to allow the cells to attach.

Test Agent Preparation: For each single agent or combination of test agents, the top concentration mixture (2× final treatment concentration) was made in sterile 1.5 ml microcentrifuge tubes and then directly transferred to the first well of the treatment dilution plates.

Tris(8-quinolinolato)gallium(III) was obtained from Niiki Pharma, Inc. Temozolomide was manufactured by the Schering Corporation and supplied in an amber glass vial. It was stored in the dark at room temperature and sealed with Parafilm® to limit exposure to light and humidity. Temozolomide (20.7 mg) was weighed out and a 400 mM white, cloudy suspension was made by adding 149 μL of 100% DMSO and brief sonication (˜10-20 seconds) in a sonicating water bath without heat.

The antiproliferative activity of the test agents was evaluated using the MTT Cell Proliferation Assay Kit (ATCC catalog #30-1010K). Cells in the log phase of growth were seeded at the indicated density into 96-well culture treated plates in 0.1 mL of complete media in all wells except for one column reserved for the media only control. The cells were allowed to attach during an overnight incubation prior to treating with test agents. Test agents were serially diluted in complete culture media (+1% DMSO where appropriate) and added to each well in a volume of 0.1 mL for a total final volume of 0.2 mL/well (0.5% DMSO final, where used). Cells were exposed to test agents for 72 hours. Following the exposure to test agents, 0.1 mL of culture supernatant was carefully removed from all wells of each plate and 0.01 mL of MTT reagent was added to each well. The plates were returned to the incubator for four hours. Following the incubation period, kit supplied detergent reagent (0.1 mL) was added to all wells. The plates were wrapped in plastic wrap to prevent evaporation and allowed to sit at room temperature in the dark overnight. The absorbance at 570 nm was measured the following day using a SpectraMAX Plus plate reader (Molecular Devices). Absorbance values were converted to Percent of Control and plotted against test agent concentrations for EC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration vs. Percent of Control were analyzed using the 4-parameter equation to obtain EC₅₀ values and other parameters that describe the sigmoidal dose response curve.

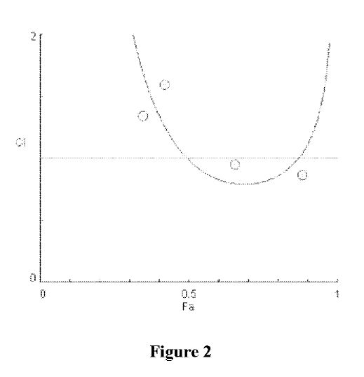

Combination data was analyzed using CompuSyn® software to calculate Combination Index (CI) values to assess synergy. The Fractional Affect (Fa) was calculated from the Percent of Control (from SoftMax® Pro) using the formula: 1-(Percent Control/100). The dosage, fractional affect and molar ratio of compounds tested in combination were entered into the CompuSyn® software for evaluation of the presence/absence of synergy. CompuSyn® assigns a Combination Index (CI) value which rates the level of compounds' affect on proliferation. CI values below 1 indicate the presence of synergy and CI values above 1 indicate antagonism. CI values close to 1 indicate an additive affect. See Chou, Pharmacol. Rev. 58(3):621-81 (2006). The combination of tris(8-quinolinolato)gallium(III) and temozolomide had a CI value of 0.812 in U251 cells, indicating a synergistic combination. FIG. 2 is a combination index plot illustrating the additive to synergistic activity between tris(8-quinolinolato)gallium(III) and temozolomide in the glioblastoma cell line U251.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

What is claimed is:

1. A method of treating brain cancer comprising administering to a patient in need of treatment a therapeutically effective amount of a compound of Formula (I) or a pharmaceutically acceptable salt thereof 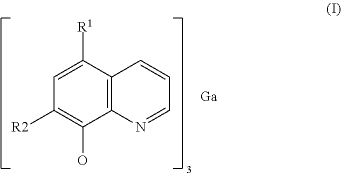
wherein R¹ represents hydrogen, a halogen or a sulfono group SO₃M, in which M is a metal ion, and R² represents hydrogen, or R¹ is Cl and R² is I.

2. The method of claim 1, said compound is tris(8-quinolinolato)gallium(III).

3. The method of claim 2, wherein said brain cancer is not glioblastoma.

4. The method of claim 2, wherein said brain cancer is astrocytoma.

5. The method of claim 2, wherein said brain cancer is glioblastoma.

6. The method of claim 2, wherein said brain cancer is a refractory brain cancer.

7. The method of claim 2, wherein said brain cancer was previously treated with BCNU.

8. The method of claim 7, further comprising administering to said patient a therapeutically effective amount of temozolomide.

9. The method of claim 2, wherein said brain cancer was previously treated with temozolomide.

10. The method of claim 9, further comprising administering to said patient a therapeutically effective amount of temozolomide.

11. The method according to claims 2, further comprising administering to said patient a therapeutically effective amount of temozolomide.

12. A kit comprising:
an effective amount of a compound of Formula (I) 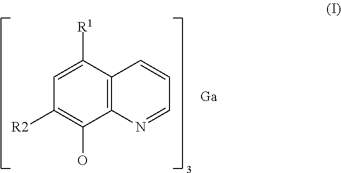
wherein R¹ represents hydrogen, a halogen or a sulfono group SO₃M, in which M is a metal ion, and R² represents hydrogen, or R¹ is Cl and R² is I, or a pharmaceutically acceptable salt thereof; and
an effective amount of temozolomide.

13. The kit of claim 12, wherein said compound is tris(8-quinolinolato)gallium(III).

14. A pharmaceutical composition comprising:
an effective amount of a compound of Formula (I) 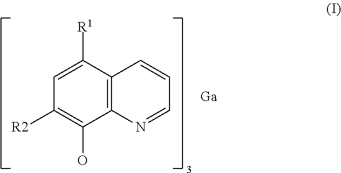
wherein R¹ represents hydrogen, a halogen or a sulfono group SO₃M, in which M is a metal ion, and R² represents hydrogen, or R¹ is Cl and R² is I, or a pharmaceutically acceptable salt thereof; and
an effective amount of temozolomide.

15. The pharmaceutical composition of claim 14, wherein said compound is tris(8-quinolinolato)gallium(III).
